(19) United States
(12) Patent Application Publication
(10) Pub. No.: US 2011/0219518 A1
(43) Pub. Date: Sep. 15, 2011

(54) Earmuffs and Designer Headband Combination

(76) Inventor: Shayna Leigh Schlickman, Highlands Ranch, CO (US)

(21) Appl. No.: 12/724,215

(22) Filed: Mar. 15, 2010

(51) Int. Cl. A61F 11/14

(52) U.S. Cl. 2/209

(57) ABSTRACT

Earmuffs and Designer Headband Combination is disclosed with a design made from a frame of thin spring like memory-flex lightweight plastic or similar resilient martial in the form of a rectangle that is curved shaped like a horseshoe frame that enables it to comfortably conform to the contour of the top and sides of the head. Just above the top of the ear a thin piece of same flexible lightweight plastic or similar resilient martial will continue to extend downwardly on the outer sides of the ear running along the bottom of the ear leaving an open square shape over each ear. The open square shape over each ear will be covered with a piece of the thin advance technology warm insulating type fabric/material, and the entire frame will be covered with the thin advance technology warm insulating type fabric/material pulled tight over the inner and outer surfaces.

### BACKGROUND OF THE INVENTION

This invention relates generally to the field of ear protection and warmth, and more specifically to an Earmuffs and Designer Headband Combination to integrate the practical function of earmuffs with the fashionable look of a headband.

Earmuffs have been around since the late 1800s to keep ears warm as well as protecting the ears from the cold and wind. It is important to protect ears from the cold, wind and harsh increment weather. Many individuals experience a painful earache when ears are exposed to cold weather for a length of time, particularly on humid or windy days. Cold weather earaches are caused by a change in the air pressure within the part of the ear called the Eustachian tube, the channel between the outer ear and the inner ear. The tube normally remains closed off unless a muscular force prompts it to open. When air pressure changes because of a sudden shift in temperature from going outside into the cold, the air trapped in the middle ear becomes less dense than the air outside, resulting in pressure within the very sensitive inner ear, causing many individuals to experience a painful cold weather earache. The best way to prevent cold weather earaches is to stabilize the temperature in the inner ear by covering up the ears before going out into the cold. Cold temperatures can also injure the exposed skin of the ears causing frost-nip or frostbite. Frost-nip may cause discomfort or temporary loss of sensation. Frostbite can damage the skin, be extremely painful and can result in the irreversible loss of tissue. Frostbite and frost-nip can be avoided by taking appropriate precautions by protecting the ears with earmuffs while out in harsh weather conditions or during extended exposure to cold temperatures.

Headbands have been a part of hairstyling for centuries and have continued to be popular throughout the ages. The slim arcuate frame allows for a wide range of possible designs elements allowing the wearer's to express individual style and incorporate with a favorite hairstyle. A headband can be used as an aesthetically pleasing accessory for the hair or for the practical use of holding a wearer's hair back away from the face.

The typical design of earmuffs in prior art include flexible plastic, metal or similar material extending over the top of the head with large round cup-like circles made up of thick padded bulky material covering the ears.

Although practical for protecting and keeping the ears warm, the bulky look of traditional earmuffs with large round circles covering the ears lack fashion appeal. The awkward appearance of most earmuff designs discourages regular use for wearers that desire a stylish look while out in the cold. The thick padding over the ears of most earmuffs tends to muffle the wearer's hearing while enjoying outdoor actives or working out in the elements. In most cases, people want to clearly hear other individuals, activities and the sounds of nature, as well as vehicles or bikes that may be approaching. The bulky circles over the ears of typical earmuffs do not allow the wearer to easily position them behind the ears when not in the cold if the user does not desire to remove and carry the earmuffs. The prior art of earmuff frames normally include multiple pieces to attach the round circle of the frame to the part of the frame that extends over the wearers ears increasing the complexity and cost of manufacture.

A headband made of fabric that encircles a wearer's head is another invention that keeps the ears warm. This headband design has a slim profile. However, some versions are made of one piece of stretchable fabric which has to be pulled over the head and dragged across the hair to be put into place, which often messes up the wearers hair. Another version of this headband is made from fabric which has two ends with velcro, fasteners or ties which can be put into place to encircle the head without messing up the wearers hair. However, strands of hair can become caught or tangled in the velcro, fasteners or ties. Many fabric type headbands that encircle the head can be tricky to put on when wearing the hair down and tend to ride up in the back of the head, matt and tangle the hair at the nape of the neck or slip out of place such that the wearer may find that they need to continually adjust the band to keep it in the desired position. The fabric headbands lack the thin arcuate spring-like memory-flex, lightweight plastic or similar resilient material frame which allows the wearer to conveniently stretch the band away from the head to be moved into the desired position and be incorporated with any hairstyle, or removed without messing up the hairstyle or tangling the hair.

Additionally there are hats that keep the ears warm. However, many will completely matt down the hair, destroy a hairstyle, cause static when removed and do not always cover the entire ear.

### BRIEF SUMMARY OF THE INVENTION

A primary object of the invention is to integrate the practical functionality of earmuffs with the fashionable look of a headband.

Another object of the invention is to combine the need to protect the ears from the cold and the want for a fashion accessory for the hair.

Another object of the invention is to utilize advance technology that offers outstanding warm insulating wind-proof fabrics and materials that are very thin, which will eliminate the bulky look and large padded circles like that of traditional earmuffs.

Yet another object of the invention is to provide a thin arcuate spring like memory-flex lightweight plastic or similar resilient martial frame that allows the wearer to conveniently stretch the band away from the head to be moved into the desired position with any hairstyle, or removed without messing up or tangling the hair.

Still another object of the invention is to create a simple design frame to allow for ease and reduced cost manufacturing.

A still further object of the invention is to allow a thin profile which makes it aesthetically pleasing and more desirable to incorporate as a chic accessory.

Still yet another object of the invention is to provide a headband-like design which will allow the wearer to easily incorporate the latest hair trends, arrange the hair in a variety of styles and includes the decorative fun designs elements of a headband while keeping the ears protected and warm.

In accordance with a preferred embodiment of the invention, there is disclosed earmuffs with a designer headband that comprises a design made from a frame of thin spring like memory-flex lightweight plastic or similar resilient martial in the general form of a rectangle that is curved to be shaped like a horseshoe or c-shape that enables it to comfortably conform to the contour of the top and sides of the head. Just above the top of the ear, a thin piece of the same flexible lightweight plastic or similar resilient martial extends downwardly on the outer sides of the ear and run along the bottom of the ear leaving a open generally square shape over each ear. The open general square shape over each ear is covered with a piece of the thin advance technology warm insulating type fabric or material, and the entire frame is covered with the thin advance technology warm insulating type fabric/material pulled tight over the inner and outer surfaces.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of a preferred embodiment of the present invention conforming to the contour of the top and sides of the head extending over the ears.

FIG. 2 is a perspective view of the plan view of the invention of FIG. 1.

FIG. 3 is a perspective view of a preferred embodiment of the arcuate frame as used in the present invention.

FIG. 4 is a perspective view of alternate embodiments of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

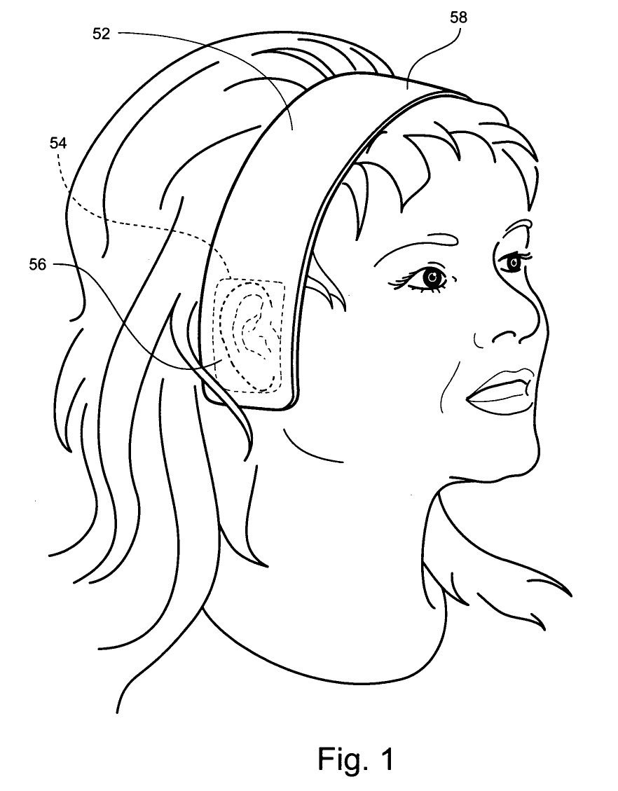

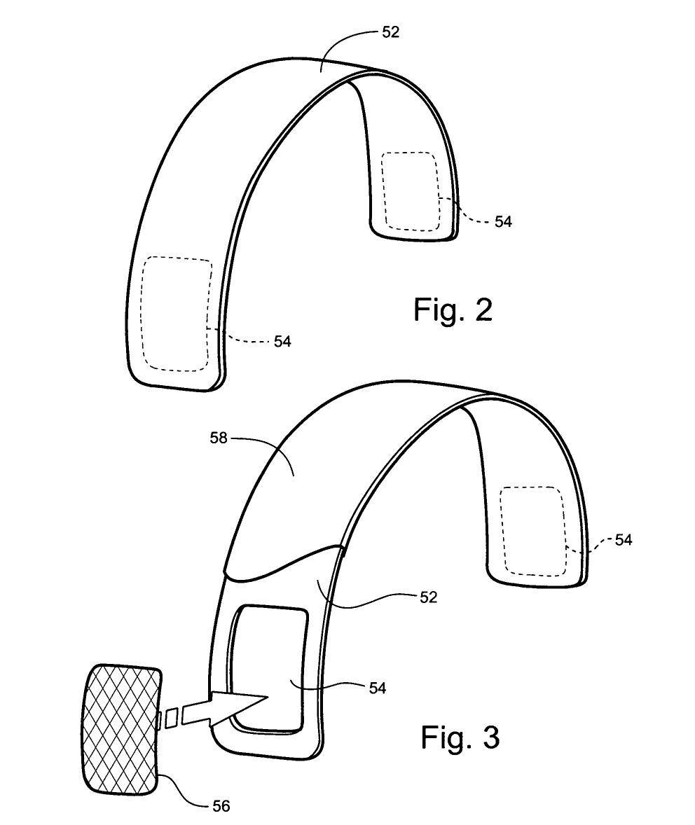

To accomplish the thin streamline design of the invention, there is shown in FIG. 3 a fabric or material overlay 58 that utilize advance technology that offers outstanding warm insulating wind-proof fabrics/materials that is very thin, which will eliminate the bulky round circles over the ears of traditional earmuffs. In the preferred embodiment of FIG. 3 there is shown earmuffs with designer headband combination having a curved shaped frame like a horseshoe or c-shape 52 made from a thin spring like memory-flex lightweight plastic or similar resilient martial generally rectangular shape frame that allows comfortable conformity to the contour of the top and sides of the head. The spring like action of the frame allows the invention to be stretched away from the head and easily moved into the desired position on the head or removed without messing up the hair. Upon further inspection of FIG. 3 it will be seen that just above where the top of the wearer's ears would reside the frame continues to extend downwardly on the outer sides of the ear running along the bottom of the ear forming an open generally square shape 54 over each ear. The open generally square shape 54 over each ear is covered with a generally square piece 56 of said advance technology fabrics or materials by means of glue or other adhesion process leaving just material over the ears for warmth, to add comfort and allows the wearer to easily hear through them. In carrying out the invention the entire frame will be covered with said advance technology type fabric or material 58 pulled tight over the inner and outer surfaces. In the particular illustration shown in FIG. 1, the invention as worn on the head shows the thin headband like design, which allows the wearer to easily incorporate with select hairstyle.

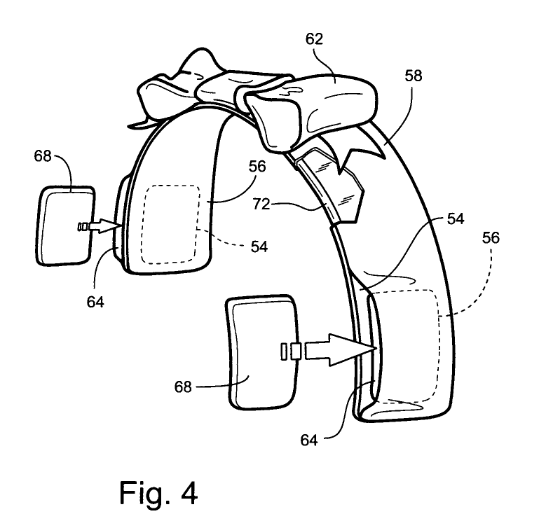

In accordance with the further aspect of the invention, FIG. 4 provides alternative embodiments such as the design may be fabricated with an additional layer of luxurious fashionable fabric or materials over said advance technology fabric/material layer 58. Such fabric may include, but not be limited to, cashmere, silk, suede or faux fur allowing for a more sophisticated stylish look. In addition, optional decorative ornamentation 62 can be attached to the outer layer of fabric such as bows, flowers, semi-precious gems, beads; the design elements are endless and can be customize to fit current trends. Preferably, select decorative ornamentation 62 can easily be detachable or removable to allow for multiple looks to add versatility and express individual style. Further, a small pocket within the advance technology fabric/material will be formed over each ear 64 that will allow for the option of inserting heat packets 68 into a small slit on the inside edge the frame within the fabric/material to provide additional warmth for extreme cold temperatures and harsh weather condition. Additionally, for the purpose of comfort, a thin layer of padding using a gel, foam, extra fabric or other cushioning material 72 may line the outer edge of the inside portion of the frame. It is contemplated that the invention will be produced in various sizes to accommodate and conform to both children and adult typical head shapes.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Earmuffs with Designer Headband Combination comprising:
a thin spring like memory-flex lightweight plastic or similar resilient martial in the form of a general rectangle that is generally horseshoe shaped frame that enables it to comfortably conform to the contour of a human head;
a thin piece of flexible lightweight resilient martial continues downward on the outer sides and along the bottom leaving a open generally square shape;
the open square shape will be covered with a piece of the thin advance technology warm insulating type fabric/material; and
a thin advance technology warm insulating type fabric/material cover pulled tight over the inner and outer surfaces.
